(19) United States
(12) Patent Application Publication
(10) Pub. No.: US 2003/0082632 A1
(43) Pub. Date: May 1, 2003

(54) Assay method and apparatus

(75) Inventor: Christopher Shumate, Encinitas, CA (US)

Correspondence Address: STEPHANIE L. SEIDMAN, HELLER EHRMAN WHITE & MCAULIFFE LLP, 7TH FLOOR, 4350 LA JOLLA VILLAGE DRIVE, SAN DIEGO, CA, 92122-1246, US

(73) Assignee: Cytoprint, Inc.

(21) Appl. No.: 10/202,969

(22) Filed: Jul. 23, 2002

Related U.S. Application Data
(60) Provisional application No. 60/347,709, filed on Oct. 25, 2001.

(51) Int. Cl. G01N 33/48

(52) U.S. Cl. 435/7.1; 422/58; 436/63; 436/29; 422/102

(57) ABSTRACT

Apparatus and methods are provided herein for conducting processes, such as biological and biochemical processes, that provide a fluid environment in which such processes can be conducted in the presence of a substantially laminar flow of the fluid through the environment. Elements of the processes, such biological or biochemical entities, can be maintained in the substantially laminar flow of fluid with minimal disruption during the processes.

### REFERENCE TO PRIORITY DOCUMENT

Benefit of priority is claimed under 35 U.S.C. 119(e) to U.S. Provisional Application No. 60/347,709 filed Oct. 25, 2001, to Chris Shumate, entitled “Assay Method and Apparatus.” The subject matter of the above-referenced provisional application is incorporated by reference in its entirety.

### BACKGROUND

The devices and methods provided herein relate to assays and tests and their applications to the fields of cell biology, molecular biology, biochemistry, chemistry, biomedical research, and other fields.

The ability to observe and monitor biological processes can be applied to a number of purposes, including drug screening and the identification of traits encoded by genes. For example, in cell-based assays, the behavior of living cells and changes in cell behavior in response to various conditions and agents may be observed. One method for observing cell behavior involves imaging. Imaging permits visualization of microscopic processes through the use of agents that provide detectable signals representative of cellular components or events. For example, a cell may be contacted with a stain that marks the location of a particular protein or that correlates with the amount and/or movement of ions in the cell. Stains introduced into the cell culture facilitate imaging of the cells and detection of changes in biological activity. Significantly, cell-based assays provide a system for evaluating the functional consequences of various conditions and treatments. Thus, a culture of cells can be exposed to a chemical compound(s) and observed for changes that may occur in the biological activity of the cells in response to the treatment.

One method for conducting cell-based imaging assays involves dispensing cells into the wells of microtiter plates to be viewed through a microscope. In automated versions of these assays, robotic liquid handling processes may be used to dispense cells and fluids into the wells and the plate may be moved for viewing of cells in the different wells using automatic indexing stages. Processing of the cells as needed to conduct the assay can involve “washing” of the cells multiple times. For example, washing is used to remove the free stain (i.e., stain not bound to a target in the cell) from bound stain incorporated into the cells so that the free stain does not obscure the detection of bound stain in the imaging process. In a manual procedure, washing can be conducted by carefully removing fluid from the wells through suctioning devices. Care must be used to avoid disturbing the cells in each well that may have settled to the well bottom. In automated procedures, a microplate washer can be used which removes, by aspiration, some of the contents of the well through one cannula and refills the well with another fluid through a second cannula.

Such methods of washing cells are associated with a number of disadvantages. For example, the high liquid flows caused by the procedure tends to detach the cells, particularly loosely adherent cells, and introduces the cells into suspension whereby they are removed from the well in the aspiration process. Cells that are undergoing a transition, perhaps as a reaction to a meaningful chemical challenge, can become less adherent to the bottom of the flow chamber or partially detach from the bottom of the flow chamber. Often, the less adherent cells are those cells exhibiting the response that the scientist wants most to observe in the assay process. Less adherent cells may be detached, introduced into suspension, and washed out of the chamber by fluid flow which is turbulent or not substantially laminar. As a result, many of the cells are lost and unavailable for the assay.

Furthermore, the washing process generally does not achieve a complete removal and replacement of fluid in the wells. In addition, the required repetition of the aspiration and dispense steps in the wash procedure involves the use of a significant amount of reagents, which can be costly as well as time consuming.

In view of the foregoing, there is a need for devices for assays, such as biochemical and/or cellular assays, that provide for flow of fluid throughout the device so that nominally adhering substances, such as cells, are not disturbed and/or detached in the process of fluid flow.

### SUMMARY

Apparatus and methods are provided herein for conducting processes, such as biological and biochemical processes, that provide a fluid environment in which such processes can be conducted in the presence of a substantially laminar flow of the fluid through the environment. Elements of the processes, such biological or biochemical entities, can be maintained in the substantially laminar flow of fluid with minimal disruption during the processes.

Disclosed is a flow chamber that includes a fluid enclosure having one or more walls that are disposed about and substantially enclose a chamber portion through which fluid can flow along a flow path. The flow chamber has an inlet port in fluid communication with the chamber portion and an outlet port in fluid communication with the chamber portion. The chamber portion has a cross-sectional area along a plane that is orthogonal to the fluid flow path. The cross-sectional area of the chamber portion is constant or changes smoothly and continuously moving along the flow path. At least dimension of the cross-sectional area is greater than about 500 μm.

Other features and advantages of the disclosed devices and methods should be apparent from the following description.

### BRIEF DESCRIPTION OF THE DRAWINGS

FIG. 1 is a side-view of a flow cell that includes a flow chamber.

FIG. 2 is a top view of the flow cell in FIG. 1.

FIG. 3 is a view of the access port of a flow chamber in FIG. 1.

FIG. 4 is a longitudinal sectional view of an alternative embodiment of a flow cell.

FIG. 5 is an alternative embodiment of a single flow cell with access and exit ports.

FIG. 6 is a side view of the flow cell in FIG. 5.

FIG. 7 is a top view of the flow cell in FIG. 5 with the access port shown.

FIG. 8 is a plate with an array of flow chambers.

FIG. 9 is an exploded view of an array of flow chambers with a chamber plate and upper and lower sealing films.

FIG. 10 is a plate with an array of waste adapters.

FIG. 11 is a side view of an alternative embodiment of a flow cell having waste collection site.

FIG. 12 is a top view of the flow cell of FIG. 11.

### DETAILED DESCRIPTION

A. Definitions

B. Flow Chamber

1. Design of a Flow Chamber

2. Preparation of a Flow Chamber

3. Use of a Flow Chamber

C. Apparatus Containing a Plurality of Flow Chambers

1. Preparation of an Apparatus Containing a Plurality of Flow Chambers

2. Use of an Apparatus Containing a Plurality of Flow Chambers

D. Methods of Use of a Flow Chamber

1. Cell Imaging Assay

2. Magnetic Particle Assay

3. ELISA-based Assay

4. Nucleic Acid Assay

5. Cell Culture Assay

6. Transfection Assay

7. Cell-based Assay

A. Definitions

Unless defined otherwise, all technical and scientific terms used herein have the same meaning as is commonly understood by one of skill in the art to which this invention belongs. All patents, patent applications, published patent applications and publications referred to herein are, unless noted otherwise, incorporated by reference in their entirety. In the event a definition in this section is not consistent with definitions elsewhere, the definition set forth in this section will control.

As used herein, a flow chamber refers to an enclosure that defines an interior space or cavity which can contain a fluid and through which a fluid can flow.

As used herein, a fluid is a substance that has the ability to flow and to conform to the boundaries of a container in which the substance is placed. Fluids include liquid and gas substances. Fluids include, but are not limited to, water, buffer, blood, preparative fluids, culture medium, reagents, organic solutions, inorganic solutions, and any fluid that can be used to conduct an assay.

As used herein, a preparative fluid is any fluid that can be used to carry out any assays or methods and, in particular, assays and methods provided herein.

As used herein, a flow path is the path of flow of a fluid. A flow path can be the path of flow of a fluid through a flow chamber. For example, a flow path can be the path of flow of a fluid from an inlet port, through the interior of a chamber and to an outlet port.

As used herein, laminar flow, with reference to a fluid, is substantially non-turbulent flow of a fluid. The degree of laminar flow can be characterized by the Reynolds number, which is a measurement of the tendency for turbulence to occur as described by the equation:

$$Re = \frac{vd ρ}{μ}$$

wherein Re is a Reynolds number, v is the average linear flow rate, d is a unit of length, ρ is the density of the fluid and μ is viscosity of the fluid. Turbulence in fluid flow occurs in flows characterized by high Reynolds (Re) numbers. Generally, turbulence in flow occurs for Re greater than or equal to about 2000. Thus, laminar flow generally can be characterized by an Re less than about 2000. In particular embodiments, laminar flow can be characterized by an Re less than about 1000, or less than about 500, or less than about 250, or less than about 100, or less than about 50, or less than about 25, or less than about 20 or less than about 10. Such laminar flows may further have an Re greater than about 1. Fluid flows with Re less than about 1, known as creeping flows, are symmetric and reversible (see, e.g., U.S. Pat. No. 6,065,864). Laminar flow can also refer to the flow of a viscous fluid in which particles of the fluid move in parallel layers, each of which has a constant velocity but is in motion relative to its neighboring layers. Laminar flow can be a streamlined, uniform flow of fluid near a solid boundary without any significant turbulence (e.g., Re less than about 2000 and greater than about 1). A fluid path that has little to no bending, little to no sharp turns, little to no steep slopes, little to no recirculation paths, little to no retroflow, little to no eddy currents, and little to no whorls generally can provide for substantially laminar flow. The term is also used to describe a linear flow of fluid that has a controllable linear velocity at the surface of a flow chamber provided and that is non-disruptive to an element (e.g., a living cell or an analyte) of a process, e.g., a biological or biochemical process, in the flow chamber.

As used herein, substantially laminar flow, with reference to a fluid, is a flow of fluid that is at least about 80% laminar, at least about 85% laminar, at least about 90% laminar, at least about 95% laminar, at least about 96% laminar, at least about 97% laminar, at least about 98% laminar, at least about 99% laminar, or at least about 100% laminar.

As used herein, “smoothly and continuously,” with reference to a flow path or the dimensions or cross-sectional area of a flow chamber, means uninterrupted and not suddenly altered, just as a smooth and continuous mathematical function is uninterrupted and not suddenly altered. A “cross sectional area that changes smoothly and continuously along a flow path,” with reference to a flow chamber, can be a feature of a flow chamber that can provide for substantially non-turbulent, laminar flow of fluid through the chamber. A flow chamber having a cross-sectional area that changes smoothly and continuously along a flow path of the chamber generally will have no abrupt transitions in the transverse cross sectional area of the flow chamber relative to the flow path. Such a chamber generally will have no abrupt changes or discontinuities in the cross sectional area of the flow chamber orthogonal to the flow path. Examples of shapes of flow chambers having a “cross sectional area that changes smoothly and continuously along a flow path” include, but are not limited to, spherical, hemispherical, bell-shaped, and oblong.

As used herein, a “cross sectional area that is constant along a flow path,” with reference to a flow chamber, means that there are no changes in the transverse cross sectional area relative to the flow path through the chamber and no changes in the cross sectional area orthogonal to the flow path. A “cross sectional area that is constant along a flow path,” with reference to a flow chamber, can be a feature of a flow chamber that can provide for substantially non-turbulent, laminar flow of fluid through the chamber. Examples of shapes of flow chambers having a “cross sectional area that is constant along a flow path” include, but are not limited to, a tube, a channel, cylinder, and a circle. In one aspect, a cross sectional area of a flow chamber can be constant along the fluid flow path of the chamber when the tangent to the surface normal to the fluid flow path is not more than about 0 degrees. A “cross sectional area that is constant or changes smoothly and continuously along a flow path,” with reference to a flow chamber, can refer to the cross sectional area transverse, or orthogonal, to the fluid flow path such that the fluid flow path therein or therethrough is smooth and continuous as described herein.

As used herein, “continuous,” with reference to fluid flow, refers to an unbroken or contiguous stream of the fluid. For example, a continuous flow may include a constant fluid flow having a set velocity, or alternatively, a fluid flow with a varying flow rate or which includes pauses in the flow rate, such that the pause does not otherwise interrupt the flow stream.

As used herein, a shear force is a force tending to cause deformation or movement of material by slippage along a plane or planes parallel to an imposed stress. Shear force along the walls and floor of a chamber caused by fluid flow through the chamber can dislodge cells from the walls and floor and cause them to be removed from the chamber.

As used herein, an eddy current is a whirl or circular current of fluid running contrary to the general flow or a deviation in the steady flow of a fluid, causing a vortex.

As used herein, retroflow is a current of fluid running contrary to the general flow of a fluid. For example, retroflow occurs in an eddy or vortex.

As used herein, a cell is a small membrane-bounded compartment containing a concentrated aqueous solution of chemicals. Cells are the basic unit of life. In particular, cells can be prokaryotic (e.g., without a well-defined nucleus) or eukaryotic (e.g., with a well-defined nucleus). Cells can be, but are not limited to, plant cells, animal cells, fungal and bacterial cells. Cells include cells of any species of organism, e.g., mammalian cells, transformed primary cells, primary cells and cell lines.

As used herein, cell growth refers to an increase in the size, form or complexity of a cell.

As used herein, cell proliferation refers to an increase in cell number by cell division.

As used herein, cell differentiation refers to a process by which cells change from a less specialized to a more specialized state usually associated with different functional roles and the expression of new and different traits.

As used herein, cell interaction refers to an alteration of cell behavior such as movement, growth, proliferation, or differentiation in response to the presence and/or action of nearby cells of the same or different type.

As used herein, a culture of cells refers to a collection of cells, typically similar or related cells, including, for example, cells that are all derived through division of cells from a common initial cell. A culture of cells can contain greater than about 2, greater than about 3, greater than about 4, greater than about 5, greater than about 6, greater than about 7, greater than about 8, greater than about 9, greater than about 10, greater than about 20, greater than about 30, greater than about 40, greater than about 50, greater than about 60, greater than about 70, greater than about 80, greater than about 90 or greater than about 100 cells. A typical 96-well cell-based assay can have 10,000 cells and a 384-well assay can have 2000 cells.

As used herein a colony of cells refers to an association of cells or unicellular organisms.

As used herein, a tissue refers to a mesh or network of cells with their intercellular substance forming a structured or organized tissue. Tissues are cooperative assemblies of cells in which the cells are usually in contact with a network of secreted extracellular macromolecules known as the extracellular matrix which helps to hold the cells together. Cells in tissues can also be held together by direct cell-cell adhesions.

As used herein transfection refers to the introduction of foreign DNA molecule into a eukaryotic cell. The introduction can be followed by expression of the introduced foreign DNA molecules.

As used herein, an analyte refers to a substance that is viewed, analyzed, assayed, screened, grown, or imaged and includes, but is not limited to, cells, such as, but not limited to, mammalian cells, microbial cells, plant cells, insect cells, animal cells, yeast cells, artificial cells, recombinant cells, blood cells, human cells, transgenic cells, genetically engineered cells, transformed cells, fibroblastic cells, epithelial cells, melanoma cells, stem cells, anchorage-dependent cells, anchorage-independent cells, tissues, and multi-cellular organisms; lipids, crystals, magnetic beads, proteins, carbohydrates, polymers, nucleic acids, and polysaccharides.

As used herein, a ligand refers to a molecule that is specifically recognized by a particular receptor or that binds to a specific site on a protein or any other molecule. Examples of ligands, include, but are not limited to, agonists and antagonists for cell membrane receptors, toxins and venoms, viral epitopes, hormones (e.g., steroids), hormone receptors, opiates, peptides, enzymes, enzyme substrates, cofactors, drugs, lectins, sugars, oligonucleotides, nucleic acids, oligosaccharides, proteins, and monoclonal antibodies.

As used herein, ELISA is Enzyme-linked immunosorbent assay and can be used quantitate the amount of antigen, proteins, or other molecules of interest in a sample.

As used herein, cellular respiration refers to the metabolic processes in a living cell which are dependent upon availability of oxygen to the cell, to generate energy for cellular growth and other functions, and the removal and balancing of carbon dioxide in the immediate environment of the cell for maintaining normal pH within the cell

As used herein, a biochemical system refers to a chemical interaction that involves molecules of the type generally found within living organisms. Such interactions include the full range of catabolic and anabolic reactions which occur in living systems including enzymatic, binding, signaling and other reactions. Further, biochemical systems, as defined herein, will also include model systems which are mimetic of a particular biochemical interaction. Examples of biochemical systems include, but are not limited to, e.g., receptor-ligand interactions, enzyme-substrate interactions, cellular signaling pathways, transport reactions involving model barrier systems (e.g., cells or membrane fractions) for bioavailability screening, and a variety of other general systems.

As used herein, test compound refers to a substance to be screened for the ability to affect a particular biochemical system. Test compounds may include a wide variety of different compounds, including chemical compounds, mixtures of chemical compounds, e.g., polysaccharides, small organic or inorganic molecules, biological macromolecules, e.g., peptides, proteins, nucleic acids, or an extract made from biological materials such as bacteria, plants, fungi, or animal cells or tissues, naturally occurring or synthetic compositions. Depending upon the particular embodiment being practiced, the test compounds may be provided, e.g., injected, free in solution, or may be attached to a carrier, or a solid support, e.g., beads. A number of suitable solid supports may be employed for immobilization of the test compounds. Examples of suitable solid supports include agarose, cellulose, dextran (commercially available as, i.e., Sephadex, Sepharose) carboxymethyl cellulose, polystyrene, polyethylene glycol (PEG), filter paper, nitrocellulose, ion exchange resins, plastic films, glass beads, polyaminemethylvinylether maleic acid copolymer, amino acid copolymer, ethylene-maleic acid copolymer, nylon, silk, etc. Additionally, with respect to the methods and apparatus provided herein, test compounds may be screened individually, or in groups. Group screening is particularly useful where hit rates for effective test compounds are expected to be low such that one would not expect more than one positive result for a given group. For example, test compounds may be screened for their ability to block systems that are responsible, at least in part, for the onset of disease or for the occurrence of particular symptoms of diseases, including, e.g., hereditary diseases, cancer, bacterial or viral infections and the like. Compounds which show promising results in these screening assay methods can then be subjected to further testing to identify effective pharmacological agents for the treatment of disease or symptoms of a disease. Alternatively, compounds can be screened for their ability to stimulate, enhance or otherwise induce biochemical systems whose function is believed to be desirable, e.g., to remedy existing deficiencies in a patient. Test compounds can also be screened for their effect on cellular response.

As used herein, cell imaging refers to the use of microscopy techniques for visualization of the microstructure (e.g., molecular and structural components) of cells and tissues. Cell imaging can be performed with light microscopy and fluorescence microscopy. Fluorescence microscopy methods provide high spatial and temporal resolution of microstructures by using florescent labels of these microstructures. Microscopy techniques can include, for example, wide-field, confocal, multiphoton excitation, fluorescence resonance energy transfer (FRET), lifetime imaging (FLIM), spectral imaging, fluorescence recovery after photobleaching (FRAP), optical tweezers, total internal reflection, high spatial resolution atomic force microscopy (AFM), and bioluminescence imaging for gene expression. These techniques can be used in various biological applications, including, for example, calcium, pH, membrane potential, mitochondrial signaling, protein-protein interactions under various physiological conditions, and deep tissue imaging. Resulting images can be obtained and recorded on film or in digital formats.

As used herein, a biopolymer includes, but is not limited to, nucleic acid, proteins, polysaccharides, lipids and other macromolecules. Nucleic acids include DNA, RNA, and fragments and analogs thereof. Nucleic acid sequences may be derived from genomic DNA, RNA, mitochondrial nucleic acid, chloroplast nucleic acid and other organelles with separate genetic material.

As used herein, proteins are complex, three-dimensional substances comprising one or more folded polypeptide chains. These chains, in turn, consist of small chemical units called amino acids. All amino acids contain carbon, hydrogen, oxygen, and nitrogen. Some also contain sulfur. A “peptide” is a compound that includes two or more amino acids. The amino acids link together in a line to form a peptide chain. There are 20 different naturally occurring amino acids involved in the biological production of peptides, and any number of them may be linked in any order to form a peptide chain. The naturally occurring amino acids employed in the biological production of peptides all have the L-configuration. Synthetic peptides can be prepared employing conventional synthetic methods, utilizing L-amino acids, D-amino acids, or various combinations of amino acids of the two different configurations. Some peptide chains contain only a few amino acid units. Short peptide chains, e.g., having less than ten amino acid units, are sometimes referred to as “oligopeptides”, where the prefix “oligo” signifies “few”. Other peptide chains contain a large number of amino acid units, e.g., up to 100 or more, and are referred to a “polypeptides”, where the prefix “poly” signifies “many”. Still other peptide chains, containing a fixed number of amino acid units are referred to using a prefix that signifies the fixed number of units in the chain, e.g., an octapeptide, where the prefix “octa” signifies eight. (By convention, a “polypeptide” may be considered as any peptide chain containing three or more amino acids, whereas an “oligopeptide” is usually considered as a particular type of “short” polypeptide chain. Thus, as used herein, it is understood that any reference to a “polypeptide” also includes an oligopeptide. Further, any reference to a “peptide” includes polypeptides, oligopeptides, and the like.) Each different arrangement of amino acids forms a different polypeptide chain. The number of chains, and hence the number of different proteins, that can be formed is practically unlimited.

As used herein, a primary structure is one wherein the number and sequence of amino acids in the polypeptide or protein are known. The peptide linkage between each of the amino acid residues is implied, but no other forces or bonds are indicated by use of the term “primary structure”. A secondary structure refers to the extent to which a polypeptide chain possesses any helical or other stable structure. A secondary structure will thus have a set of angles (phi and psi angles) for each residue of the chain. A tertiary structure is a term used to refer to the tendency for the polypeptide to undergo extensive coiling or folding to produce a complex, somewhat rigid three-dimensional structure. A quaternary structure is a term used to define the degree of association between two or more polypeptides, e.g., between two tertiary structures, such as a target peptide and a receptor.

“Domain” structures are used to refer to well-separated parts within globular proteins, i.e., within tertiary structures. See, e.g., Linderstrom-Lang, et al., “Protein Structure and Enzyme Activity,” The Enzymes, (P. D. Boyer, Ed.), 1:443-510, Academic Press, New York (1959); and Schulz et al., Principles of Protein Structure, Springer-Verlag, New York (1984).

Those skilled in the art will recognize that the above description of a polypeptide chain and the factors that define its total structure are somewhat simplified. However, the above description nonetheless provides a sufficient background for understanding the subject matter described herein. For a more thorough description of polypeptide structure, see, e.g., Ramachandran et al., “Conformation of Polypeptides,” Adv. Prot Chem. 23, 283-437 (1968).

As used herein, a “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogues thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation. (see Sambrook et al. Molecular Cloning, A Laboratory Manual ).

As used herein, “port” with reference to a flow chamber refers to a structure that provides for communication between the interior and exterior of the chamber. Thus, a port can provide for the transfer of a fluid from the exterior of the chamber to the interior of the chamber or from the interior of the chamber to the exterior of the chamber. A port may be a structure that is attached to or extends from the main body of a flow chamber, e.g., a tube or channel, or can be only a hole in a wall of a chamber. The terms “access port” and “inlet port”, as used herein, are interchangeable and refer to a port through which a substance, e.g., a fluid, is introduced into a chamber. The terms “exit port” and “outlet port”, as used herein, are interchangeable and refer to a port through which a substance, e.g., a fluid, is removed from a chamber.

As used herein, a semi-permeable membrane refers to a bio-compatible material which is impermeable to liquids and capable of allowing the transfer of gases through it. Such gases include, but are not limited to, oxygen, water vapor, and carbon dioxide. Semi-permeable membranes are an example of a material that can be used to form a least a portion of an enclosure defining a flow chamber cavity. The semi-permeable membrane may be capable of excluding microbial contamination (e.g. the pore size is characteristically small enough to exclude the passage of microbes that can contaminate the analyte, such as cells). In a particular aspect, a semi-permeable membrane can have an optical transparency and clarity sufficient for permitting observation of an analyte, such as cells, for color, growth, size, morphology, imaging, and other purposes well known in the art.

As used herein, an interrogation surface, with reference to a flow chamber, is a part of a flow chamber that can provide for detection, assaying, or imaging of an substance contained within the chamber.

As used herein, a tissue culture medium is a liquid solution or suspension which is used to provide sufficient nutrients (e.g. vitamins, amino acids, essential nutrients, and salts) and sufficient properties (e.g. osmolarity and buffering) to maintain living cells, tissue cells, and/or multi-cellular organisms, and support their growth, development, and/or proliferation.

Commercially available tissue culture medium are available to those of skill in the art.

As used herein, the term “bind” refers to any physical attachment or close association, which may be permanent or temporary. The binding can result from hydrogen bonding, hydrophobic forces, van der Waals forces, covalent and ionic bonding.

As used herein, particles includes insoluble materials of any configuration, including, but not limited to, spherical, thread-like, brush-like, and irregular shapes. Particles can be porous with regular or random channels inside. Particles can be magnetic. Examples of particles include, but are not limited to, silica, cellulose, Sepharose beads, polystyrene (solid, porous, derivatized) beads, controlled-pore glass, gel beads, magnetic beads, sols, biological cells, subcellular particles, microorganisms (protozoans, bacteria, yeast, viruses, and other infectious agents), micelles, liposomes, cyclodextrins, and other insoluble materials.

B. Flow Chamber

Provided herein are devices that can be used to assay, screen or monitor substances, and/or conduct processes, such as, but not limited to, biological processes, including, for example, chemical processes, biochemical processes, immunological processes, and biomedical processes. Substances that can be assayed by the devices provided herein include, but are not limited to, cells, lipids, crystals, magnetic beads, proteins, carbohydrates, polymers, nucleic acids, polysaccharides, polypeptides, chromatographic materials (e.g. reverse phase and normal phase), antibodies, enzyme conjugates, catalysts, and size exclusion materials. Cells that can be analyzed, screened, grown, cultured, differentiated, and/or proliferated in the flow chambers provided herein include, but are not limited to, mammalian cells, microbial cells, plant cells, insect cells, animal cells, hepatocytes, stem cells, neurons, artificial cells, recombinant cells, blood cells, human cells, transgenic cells, genetically engineered cells, transformed cells, fibroblastic cells, epithelial cells, melanoma cells, anchorage-dependent cells, anchorage-independent cells, tissues, and multi-cellular organisms.

1. Design of Exemplary Flow Chamber

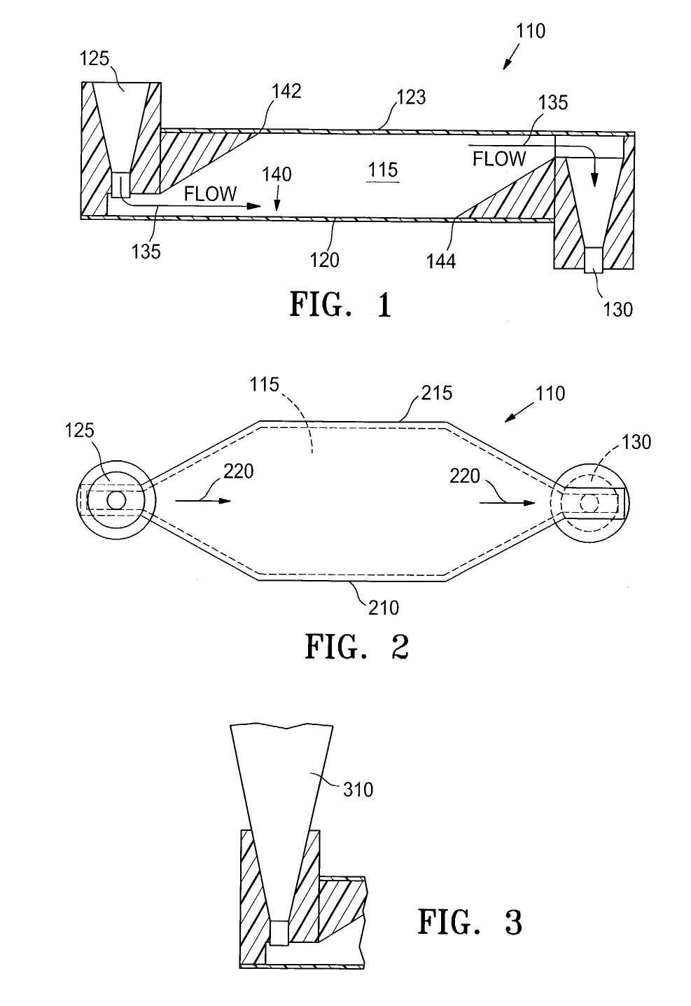

Apparatus provided herein contain one or more flow chambers configured to encourage laminar flow of a fluid through the flow chamber. FIGS. 1 and 2 show an exemplary embodiment of a flow cell 110 that can be used to assay, screen or monitor substances within a flow chamber 115 that is contained within the flow cell 110. The flow chamber 115 is formed by a set of walls, such as a bottom wall 120 and a top wall 123, as shown in the side view of FIG. 1, and a first side wall 210 and a second side wall 215, as shown in the top view of FIG. 2, that form a fluid enclosure. Although the flow chamber 115 is shown and described as being formed by four walls, it should be appreciated that more or less walls can be used to form the flow chamber 115. For example, the flow chamber 115 could be tubular such that a single, annular wall defines the chamber 115.

At least one of the walls of the flow chamber 115 comprises an interrogation surface 140 that can provide for detection, assaying, orimaging of a substance contained in the flow chamber 115. The interrogation surface 140 is described herein as being on the bottom wall 120 of the flow chamber 115, although it could also be on one of the other walls. The interrogation surface 140 does not necessarily encompass the entire flow chamber 115, but can be contained within a portion of the flow chamber 115. The portion of the flow chamber 115 that contains the interrogation surface 140 is referred to herein as the interrogation region of the flow chamber 115.

As described in detail below, a fluid can be flowed through the flow chamber 115. The interior of the flow chamber 115 has a shape that encourages laminar flow as a fluid flows through the flow chamber, particularly in the interrogation region of the flow chamber 115. The laminar flow is at least partially achieved through the shape of the walls 120, 123, 215, 215, particularly in the interrogation region of the flow chamber 115. In this regard, the walls of the flow chamber 115 have a contour that minimizes resistance to motion through a fluid that is flowing adjacent to or near the walls, which results in less likelihood of turbulent flow in the flow chamber 115. Such contours can be characterized by a smooth surface that has little or no sharp edges.

For example, FIG. 1 shows a side view of an exemplary flow chamber 115. At an entry region to the flow chamber 115, the upper wall 123 slopes upward at about a 40 degree angle relative to horizontal. Moving in the flow direction (from left to right with respect to FIG. 1), the slope of the upper wall 123 changes at a point 142 to about a zero degree slope from the 40 degree slope. This would be an example of a smooth contour. The bottom wall 120 undergoes a similar change in slope from a zero degree slope to about a forty degree slope at a point 144. It can be seen from the top view of FIG. 2 that the side walls 210, 215 also undergo similar changes in contour. Such contour changes are designed to minimize the resistance to motion of a fluid that is traveling at or near the walls.

The contours of the walls of the flow chamber 115 can also be characterized by the cross-sectional shape of the flow chamber along a plane that is orthogonal to the flow path of a fluid flowing through the flow chamber. The cross-sectional shape can change or can remain constant moving through the flow chamber 115 along the direction of the flow path. If the cross-sectional shape changes, it changes in a smooth and continuous manner that is characterized by the lack of sudden changes in the area of the cross-section.

With reference to FIGS. 1 and 2, the flow cell 110 includes at least one inlet or access port 125 and at least one outlet or exit port 130. The ports 125, 130 are both in fluid communication with the flow chamber 115. The access port 125 forms an inlet conduit that can be used to flow fluid into the flow chamber 115. The exit port 130 forms an outlet conduit that can be used to flow fluid out of the flow chamber 115. In this regard, the access port 125 and exit port 130 define an entryway and exitway, respectively, of a flow path that fluid follows as the fluid flows into, through and out of the flow chamber 115 along a flow path, which is shown using arrows 135 in FIG. 1 and arrows 220 in FIG. 2. The ports can be resealable by using materials such as, but not limited to, rubber, silicone, silicone-rubber, or other elastomeric material suitable for forming a resealable port. The access port 125 can serve as an input port for the introduction of fluids, such as by a pipette 310 (as shown in FIG. 3) by syringe, by cannula, by a pumping device, or by any method known in the art for transfer of fluids.

Fluids that can be introduced into the chamber include, but are not limited to, water, buffer, isotonic fluid, cell culture medium, cell suspensions, reagents, wash solution, preparative fluids, mixtures of fluids, organic solvents, polarity selective reagents, scavengers, reactants, blocking substances, reactive gases, or any other fluid that may be used to maintain a substance and/or conduct a process. Any existing fluid in the chamber can be displaced by fluids entering the flow chamber and can exit via the outlet port.

In one embodiment, shown in FIG. 1 and 2, the flow chamber 115 is configured so that the incoming flow of fluid from the access port 125 travels downward for a finite period through the access port 125, spreads laterally into the chamber 115, and moves across or near to the bottom wall 120 of the chamber to provide an effective, gentle rinse, of any substances, such as an analyte, adhered or partially adhered to the interrogation surface. The fluid then flows through the outlet conduit of the exit port 130. FIG. 1 shows the fluid exiting the chamber 115 at a location that is higher than the level of the chamber bottom wall 120. In another embodiment, the fluid enters the chamber 115 at a higher elevation than where the fluid exits the chamber 115. This would allow gravity to “push” the fluid through the chamber 115. In other embodiments, the fluid can enter the bottom of the chamber 115 and leave from the bottom of the chamber 115, or enter the top of the chamber 115 and leave from the bottom of the chamber 115, or enter the top of the chamber and leave from the top of the chamber. Other embodiments where there are points in the chamber 115 that are higher than the exit port and where surface tension affects the shape of the fluid path are also contemplated herein.

In this flow-through chamber design, the pipette 310, or any other tool used to introduce fluid into the chamber, can act as a dispenser and washer because the fluid dispensed into the access port 125 by the pipette 310 efficiently displaces the existing fluid within the chamber. Such a flow-through design is efficient and readily adaptable to automated processing. Reagent waste is minimized because of the efficient displacement of chamber fluids with wash fluids. In addition, the flow chamber does not require a plate lid which needs to be removed and replaced. Thus, sterility in the chamber 115 is enhanced and automation complexity is reduced because there is no lid that is removed and replaced during processing, which may compromise sterility of the contents of a well having a lid.

As mentioned, the flow chamber 115 is designed to achieve a flow of fluid (e.g. fluid that is introduced into the chamber) that is substantially laminar. Such flow does not move in all directions continually mixing within the chamber, does not recirculate throughout the chamber, does not retroflow, does not have sharp changes in the slopes of the fluid flow path, and does not disrupt or remove from the chamber substance, e.g., an analyte, that is being viewed, analyzed, or assayed. An analyte can be a substance that is viewed, analyzed, assayed, screened, grown, or imaged within the chamber and includes, but is not limited to, cells, such as, but not limited to, mammalian cells, microbial cells, plant cells, insect cells, animal cells, yeast cells, CHO cells, hepatocytes, stem cells, neurons, artificial cells, recombinant cells, blood cells, human cells, transgenic cells, genetically engineered cells, transformed cells, fibroblastic cells, epithelial cells, melanoma cells, anchorage-dependent cells, anchorage-independent cells, tissues, and multi-cellular organisms; lipids, crystals, magnetic beads, proteins, carbohydrates, polymers, nucleic acids, and polysaccharides.

The non-disruptive fluid flow within the chamber 115 will provide for little to no loss of substances, e.g., analytes, from the flow chamber 115, which will provide for higher quality assays, for improved signal to noise ratios, and, for example, in the case of cells, for better growth, differentiation, and/or proliferation of the cells. Thus, the flow of fluid through the chamber 115 is substantially laminar, with little to no retroflow, recirculation, eddy currents, whorls in the flow path, or shear forces against analytes contained in the chamber.

The flow chamber 115 can also be designed such that there are little to no pockets of dead space wherein fluid can be trapped, that there is no dead volume, and that there is no area of the chamber that does not receive fluid.

The design of the flow chamber 115 can be such that a linear flow of fluid that has a controllable linear velocity at the surface of the flow chamber can be achieved. Such a fluid flow is non-disruptive to substance, e.g., an analyte, in the flow chamber. Typically the flow path of fluid is smooth, continuous, and uninterrupted.

The design of the flow chamber 115 is such that it provides for substantially or near complete displacement of fluid when a sufficient volume of fluid is added to the chamber. The design of the chamber 115 is such that there are little to no shear forces generated by the flow that can disrupt a substance, e.g., an analyte, in the chamber 115. Thus, any force exerted on a substance, e.g., an analyte, by the fluid flow is less than any force by which the substance may adhere to the chamber. The adhesion force of a substance, e.g., an analyte, varies for different substances and based on the material of which the chamber 115 is constructed.

Design aspects of a flow chamber that can be relevant in providing for substantially laminar flow in the chamber include, but are not limited to, the placement of the inlet and exit ports 125, 130 and the geometry of the chamber. The flow chamber has at least two ports which can provide for introduction of fluid into the chamber 115 and removal of fluid from the chamber 115. Additional ports may be used for such purposes as, but not limited to, the introduction of gases and venting of the chamber (e.g. vent ports).

The inlet and outlet ports can be located at various locations in the chamber 115. For example, the ports can be opposite each other, on the same side of the chamber 115, or on top and/or bottom of the chamber 115. The inlet port 120 can be such that it provides for fluid to enter the chamber 115 at a lower elevation relative to the outlet port 130. The inlet port 125 can be such that it provides for fluid to enter the chamber 115 at a higher elevation relative to the outlet port 130. The inlet port 125 can be such that it provides for fluid to enter the bottom level or the top level of the chamber 115. The outlet port 130 can be such that it provides for fluid to exit the bottom level or the top level of the chamber 115. The inlet and outlet ports can be designed so that surface tension and/or capillary action controls the flow of fluid through the chamber 115 and so that there is no spillage of fluid from the chamber 115, provided that any pressure that may be supplied to the ports does not exceed a threshold pressure. One of skill in the art can empirically determine the threshold pressure that cannot be exceeded if is to be maintained inside the chamber 115. If present, a vent port can be designed such that it allows air to escape but blocks fluid from flowing through the chamber.

The geometry of the chamber 115 is characteristic such that it provides for a flow of fluid within the chamber 115 that is not disruptive, that does not create eddy currents or whorls, that is not recirculated, that does not have retroflow, and that is substantially laminar flow. Substantially laminar flow promotes filling and gentle rinsing of the chamber 115 in order to minimize fluid flushing time and to minimize wall shear forces which could displace analytes, such as cells, from the chamber 115. The design and geometry of the chamber 115 are characteristic such that it provides for a flow of fluid in the chamber 115 with a constant linear velocity at the surface and with minimum changes in the slope of the fluid flow path. The geometry of the chamber 115 is characteristic such that the bottom, side walls, and/or edges of the chambers are smooth and/or rounded, that the interior of the chamber 115 has smooth surfaces, and that the chamber 115 lacks properties and lacks surfaces that will result in disruptive, non-laminar, turbulent fluid flow. The geometry of the chamber 115 is also characteristic such that it does not leave significant ullage (i.e., left over air or gas bubbles) in a chamber that is not completely filled with fluid.

In addition, the geometry of the chamber 115 is such that it can allows for complete fluid replacement, efficient rinsing of the chamber 115, and a hydrostatic design which retains fluid in the chamber when desired. Thus, the geometry of the chamber 115 can be symmetric on two or all sides, cylindrical, spherical, tubular, hemispherical, circular, oblong, or any shape that will achieve non-disruptive, substantially laminar fluid flow. The design of the chamber 115 is also characteristic such that it provides for an interrogation surface to observe, assay, image, or detect a substance, e.g., an analyte, in the chamber.

Possible chamber shapes include those in which the cross sectional area of the chamber 115 is constant throughout at least a portion of the flow path, such as in the interrogation region of the chamber 115. Such shapes include, but are not limited to, cylinders, tubes, circles, a tube, a channel, a circle, and shapes that are symmetric on two or all sides. Other chamber shapes include, but are not limited to, shapes in which the cross sectional area of the chamber 115 changes smoothly and continuously along the fluid flow path in the interrogation region of the chamber. Such shapes include, but are not limited to, spherical, hemispherical, bell-shaped, and oblong shapes. The geometry of the chamber is characteristic such that there are minimum or no abrupt transitions in the transverse cross sectional area of the flow chamber 115 relative to the flow path, that there are little to no abrupt changes or discontinuities in the cross sectional area of the flow chamber 115 orthogonal to the flow path such that substantially laminar flow of fluid is produced in the flow chamber 115.

Exemplary chambers based on this design are described herein. In one embodiment, the internal configuration of the flow chamber 115 can have a transverse dimension along a longitudinal axis that tapers laterally to a smaller transverse dimension adjacent to the junction of the access and exit ports, which can facilitate laminar flow of fluid or flow of fluid which is substantially laminar into the access port, through the flow chamber, and out of the exit port. As shown in FIG. 1, in one embodiment, the access port 125 has a cylindrical shape that tapers moving downward along the access port 125. The outlet port 130 has a cylindrical shape that tapers moving downward. A proximal portion of the chamber 115 is disposed upstream from the interrogation region of the chamber 115, and a distal portion of the chamber is disposed downstream from the interrogation region.

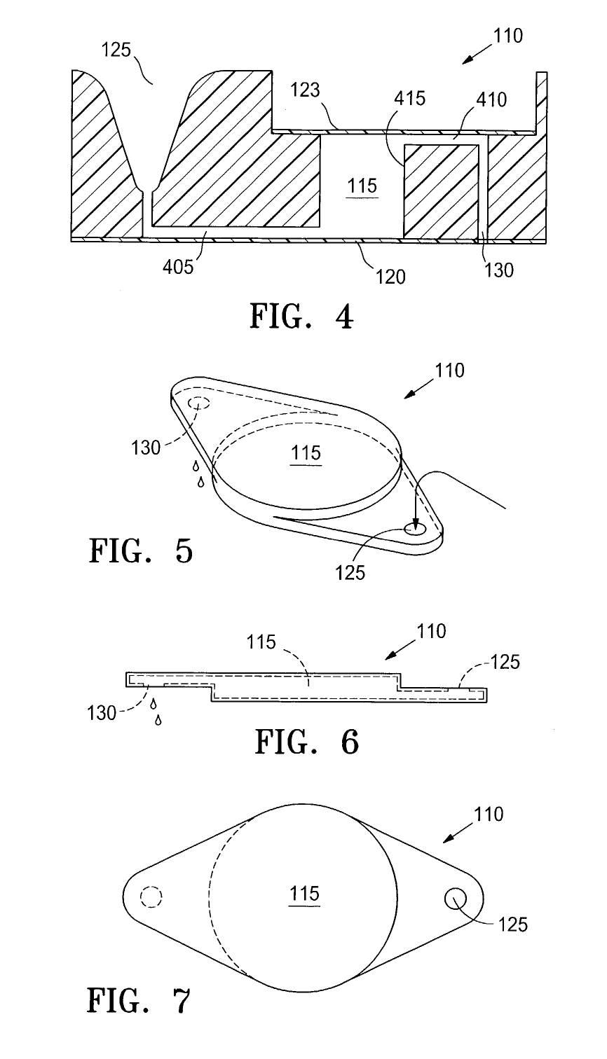

FIG. 4 shows a sectional side view of an alternate embodiment of the flow cell 110. The access port 125 has an inverted cone shape. The interrogation region of the chamber 115 has a rectangular shape. In addition, the chamber 115 has an elongate entry region 405 that is disposed adjacent to the bottom wall 120 of the chamber 115. The elongate entry region 405 has a constant cross-sectional shape moving along the flow path of fluid. The chamber 115 also includes an elongate exit region 410 that is disposed adjacent to the upper wall 123 of the chamber 115. The exit region leads to the exit port 130.

As fluid travels from the access port 125, through the chamber 115 and through the exit port 130, there can be a small area of discontinuity in flow, such as at a side wall 415 of the chamber, shown in FIG. 4. Thus, the efficiency of fluid displacement may be less than that of the flow chamber in FIG. 1. However, continuity of fluid flow may be improved by rounding the corners of the side wall 415. Thus, any turbulent flow within the flow chamber 115 of FIG. 4 is minimized such that fluid within the flow chamber 115 is substantially efficiently replaced by fluid pumped, injected or otherwise forced into the access port 125 of the flow chamber 115 and such that the flow through the flow chamber 115 is substantially laminar.

FIGS. 5-7 show an alternative embodiment of a single flow cell 110 with access and exit ports. The embodiment of the flow cell 110 shown in FIGS. 5-7 has a flow chamber 115 that has a circular shape when viewed from the top, as best shown in FIG. 7. The access port 125 is located on a first end of the flow cell 110 and the exit port 130 is located on a second end of the flow cell 110. Fluid travels into the flow cell 110 through the access port 125, which is located on an upper side of the flow cell 110, and exits through the exit port 130, which is located on a bottom side of the flow cell 110. Fluid moves circularly from the access port 125, into the flow chamber 115, and out through the exit port 130.

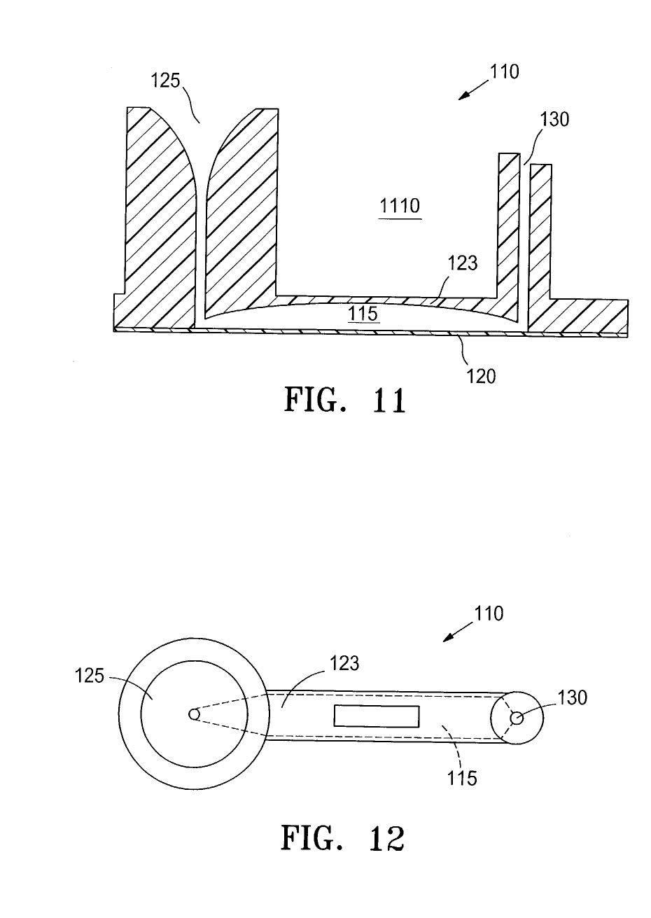

FIGS. 11 and 12 show another alternative embodiment of a single flow cell 110. The flow cell 110 shown in FIGS. 11 and 12 has a flow chamber 115 with a top wall 123 that is concave with respect to a flat bottom wall 120. The bottom wall 120 can be transparent, which can be formed from transparent material such as, but not limited to, polyolefin or any material that is biocompatible. Non-transparent material can also be used to make the bottom wall 120. The flow chamber 115 is configured so that the incoming flow of fluid from the access port 125 travels vertically downward for a finite distance, spreads laterally across the bottom of the chamber 115, and exits from the chamber 115 through the exit port 130.

As fluid exits the exit port 130, waste can collect in a sequestered or communal waste reservoir 1110 located above the chamber 115. The waste reservoir is positioned such that fluid that exits the exit port 130 can overflow and collect in the waste reservoir 1110. The flow cell 110 shown in FIGS. 11 and 12 does not require a special wash station fixture to direct the effluent to waste. The volume capacity of the waste reservoir 1110 can allow for fluid storage of, for example, 5-10 times the amount of volume of the chamber 115.

The ports and chamber walls of any of the flow chambers 115 can be prepared, for example, from an opaque, e.g. black, plastic to eliminate stray light from above the chamber. Dark lids are currently used for this purpose and require removal for liquid addition. Waste reservoirs and opaque plastic are compatible with other embodiments of the flow chamber described herein. The skilled artisan will appreciate that other designs of flow chambers with substantially laminar flow are contemplated herein. A single flow chamber, such as those provided herein, can be used alone or on a plate or support in alternative embodiments, as described more fully below. A single flow chamber can be fitted for automation using methods well known in the art.

As fluid travels from the inlet port, into the main chamber, and through the outlet port of the flow chambers provided herein, the fluid flow is substantially laminar, smooth, continuous, and uninterrupted, with little to no bending, sharp turns, retroflow, recirculation, eddy currents, or whorls in the flow path. The geometry of the flow chamber can be such that the cross sectional area is constant or changes smoothly and continuously along the flow path. The fluid flow though the chambers provided herein has a controllable linear velocity at the surface of the flow chamber and that is non-disruptive to an analyte in the flow chamber. The fluid flow though the chambers provided herein is such that there is complete displacement of fluid when a sufficient volume of fluid is added to the chamber and is such that there is no trapping of fluid within the chamber. Thus, turbulent flow within the flow chamber is minimized such that the fluid flow in the chamber is substantially laminar.

Laminar flow, or substantially laminar flow, generates minimal shear forces within the flow chamber during dispensing of fluids into the flow chamber and washing of the flow chamber. Reduced shear forces allows separation of free materials from bound materials in the flow chamber in a gentle manner and can prevent detachment of substances, such as cells, that have settled to the bottom of the flow chamber.

The walls of a flow chamber (such as the walls 120, 123, 210, 215) may be formed by a sealing film made from a transparent material such as, but not limited to, polyolefin. In some embodiments, the sealing film forms the interrogation surface of the flow chamber. Other material that can be used to make the sealing film include, but are not limited to, polystyrene, polyethylene, polycarbonate, ethylene vinyl acetate, polypropylene, polyurethane, polysulfone, polytetrafluoroethylene, petroleum based polymers, synthetic cross-linked monomers, porous glass, and any material that is biocompatible . Non-transparent material can be used to make the sealing film, particularly when interrogation of the titrant or cellular by-product is the objective of the assay and/or experiment. The choice of the sealing film may depend on the type of analyte that is assayed, tested, or grown. In an exemplary embodiment, the sealing film is made of polyolefin that has a thickness of 25-500 microns.

The sealing film can be such that it is transparent for optical imaging, for microscopy, and for fluorescence readers. The sealing film can be of sufficient optical transparency and clarity to permit observation of an analyte, such as cells. The sealing film can be detachable, can be permeable or semi-permeable to gases such as oxygen and carbon dioxide, and can permit imaging through the transparent material of the sealing film. The transparent sealing film can eliminate the need for stringent environmental requirements of live cell assays and can obviate the need for a carbon dioxide rich atmosphere. The sealing film can be such that it is impermeable to gases and non-transparent.

As discussed above, an interrogation surface, such as an image site, is disposed within the chamber 115 and is viewable through any transparent portion(s) of the chamber. At least one side of the chamber 115 can be transparent. In an alternative embodiment, one side of the chamber is transparent, and the other side is opaque. In another embodiment, one side of the chamber is transparent, and the other side is black. In another embodiment, both sides of the chamber are transparent. The image site is used for viewing of the analyte via such means as, but not limited to, microscopy, fluorescence reader, and viewing with the naked eye (e.g. to detect changes in color). The access port 125 for the flow chamber 115 can be sealed or can be open in a fashion that is compatible with the appropriate labware such as, but not limited to, pipette tips, syringe needles, pumps, and cannula, that can be used to introduce fluid into the flow chamber.

The flow cells 110 provided herein can be made from polymers, plastic, or any such suitable material, and are of a size such that at least one cross sectional dimension is greater than about 500 μm. In one embodiment, a flow chamber has a circular cross-sectional dimension of about 6.7 mm in diameter, and a rectangular cross-sectional dimension of 2 mm on each side. The flow chamber could also be oval-shaped, with the dimensions being about 1-4 mm long and 1-3 mm wide. The flow rate in the chamber can be, for example, in the range of 10 uL/min up to 100 uL/sec. The interior volume is such that a flow chamber can have an internal volume of about 1 to about 1000 μL, specifically about 20 to about 100 μL. In some embodiments, a flow chamber may have a volume of about 35 to about 45 μL. In other embodiments, a flow chamber may have an internal volume of about 15 μL to 25 μL, or about 0.1 μL to 10 μL, or any other desired volume depending upon the capacity required for a desired assay.

The size and internal volume of the flow chamber 115 can accommodate one or more cells, a culture of cells, a colony of cells, or cell tissues. Thus, the size and internal volume are such that a culture of cells can be monitored in a native environment, such as a tissue, in which neighboring cells can affect each other in terms of cellular response to drugs. The size and internal volume of the flow chamber 115 are such that a culture of cells can be assayed for sampling purposes (e.g. particularly in the case where some cells are not viable) and for resolution purposes to improve the signal to noise ratio. The size and internal volume are such that a culture of cells can be grown under conditions that promote a high rate of cell growth and density. Cells vary in size based on the cell type and are typically 10 to 20 μm in diameter (Molecular Biology of the Cell, Alberts et al. Eds., Garland Publishing, Inc. New York 1994). In some embodiments, the size of the flow chamber is about the size that is typical in the field of view of a microscope. In certain embodiments the cells are at least about greater than 50 μm², greater than 100 μm² to about 1000 μm² image within the microscope field of view.

2. Preparation of a Flow Cell

The flow cells 110 described herein can be made by injection molding using methods well known in the art. In particular, molds that are used to manufacture the flow cells 110 described herein can be prepared via injection molding or thermoforming methods. The material used to make the cells 110 can be biologically and/or chemically inert and non-toxic. Materials that can be used to make the flow cells 110 include, but are not limited to, plastic, thermoplastic, synthetic, or natural materials that can be fabricated into a flow cell 110. Other materials that can be used to make the cells 110 include, e.g., glass, quartz and silicon as well as polymeric substrates, e.g. plastics. In the case of polymeric substrates, the substrate materials may be rigid, semi-rigid, or non-rigid, opaque, semi-opaque or transparent, depending upon the use for which they are intended. For example, devices which include an optical or visual detection element, will generally be fabricated, at least in part, from transparent materials to allow, or at least, facilitate that detection. Alternatively, transparent windows of, e.g., glass or quartz, may be incorporated into the device for these types detection elements. Additionally, the polymeric materials may have linear or branched backbones, and may be crosslinked or non-crosslinked. Examples of polymeric materials include, e.g., polydimethylsiloxanes (PDMS), polyurethane, polypropylene, polyvinylchloride (PVC) polystyrene, cycloolefin, polysulfone, polycarbonate and the like.

Materials used to make the flow cells 110 provided herein can have low fluorescence or reflectance properties. Fluorescence pigments can be introduced by coating or mixing or by any means during the manufacture of the material used to make the flow chambers provided herein. The polymers used can include pigments or a mixture of pigments to darken the flow cells 110 and reduce background. The flow cells 110 can be machined, molded, assembled from layers of injection-molded plastic each of which forms part of the chamber, inlet and outlet, or made by any method well known in the art. In some embodiments, the material used to make the chambers is pretreated before and/or after assembly by means such as, but not limited to, electron beam etching, poly-lysine coating, and statically charging the materials. Pretreatements can have the intent of modifying the surface properties of some or all of the material, for example to make the surface more hydrophobic or more hydrophilic.

In some embodiments, opaque and/or black plastic is used to make the ports and chamber walls of the flow chamber. The sealing film can be made from polymers, such as, but not limited to, polystyrene, polyethylene, polycarbonate, polyolefin, ethylene vinyl acetate, polypropylene, polyurethane, polysulfone, and polytetrafluoroethylene. The choice of the composition of the sealing film will depend on the analyte to be assayed or grown or on the material of the flow chamber to be used. The sealing film can be added to the flow chamber by mechanical means, such as, but not limited to, a non-permanent locking means and a clamping means. The sealing film can be attached to the flow chamber by chemical means, such as, but not limited to, using an adhesive agent or bonding agent. Adhesive agents include, but are not limited to, double-faced adhesive tape, a polymeric adhesive, a pressure-sensitive acrylic adhesive, a hot-melt adhesive, rubber cement, or any other adhesive means known in the art. The sealing film may be attached to the flow chamber via heat bonding, sonic welding, pressure fit sealing, or a molding process.

3. Use of Single Flow Chambers

The configuration of a flow chamber 115 can provide for a laminar flow of fluid, or a flow of fluid which is substantially laminar through the flow chamber 115. Fluid may also exhibit laminar flow or flow which is substantially laminar when exiting the flow chamber through a drain port such as an exit port 130. Thus, the configuration of such a flow chamber 115 can minimize the potential for turbulent fluid flow in the flow chamber. Fluid pumped into the access port of a flow chamber 115, such as the chambers shown in FIGS. 1-7, 11, and 12, can displace the existing fluid in the chamber during injection into the access port. In some methods of using the assay devices as described herein, a fluid entering a chamber 115 of an assay device can displace a fluid within the chamber in a time period of about 2 to about 10 seconds, more specifically about 4 to about 6 seconds. For an embodiment of an assay device with a chamber having an internal volume of about 85 μL, a flow rate of about 8 μL/s to about 43 μL/s, specifically about 16 μL/s to about 18 μL/s could be used. For an embodiment of an assay device with a chamber having an internal volume of about 40 μL, a flow rate of about 4μL/s to about 20 μL/s, specifically about 7 μL/s to about 9 ρL/s could be used. Additionally, for an embodiment of an assay device with a chamber having an internal volume of about 20 μL, a flow rate of about 2 μL/s to about 10 μL/s, specifically about 3 μL/s to about 5 μL/s could be used. A flow rate of about 7 μL/s to about 35 μL/s, specifically about 13 μL/s to about 15 μL/s could be used for a chamber having an internal volume of about 70 μL.

In use, the analyte of interest as well as fluid can be introduced into the flow chamber by any means well known in the art. In particular, the inlet port 125 can be configured to receive a pipette tip, a syringe needle, a cannula, or for any labware suitable for the introduction of analyte and/or fluid. Analytes include, but are not limited to, cells, such as, but not limited to, mammalian cells, microbial cells, plant cells, insect cells, animal cells, yeast cells, artificial cells, recombinant cells, blood cells, human cells, transgenic cells, genetically engineered cells, transformed cells, fibroblastic cells, epithelial cells, melanoma cells, stem cells, anchorage-dependent cells, anchorage-independent cells, tissues, and multi-cellular organisms; lipids, crystals, magnetic beads, proteins, carbohydrates, polymers, nucleic acids, and polysaccharides.

Fluids that can be introduced into the flow chamber include, for example, water, buffer, blood, preparative fluids, culture medium, or any fluid needed to conduct an assay. Fluid flow rates can be reduced, using methods well known in the art, for analytes, such as cells, that are partially adherent or non-adherent. In one embodiment, analytes, such as cells, and fluids are added to just fill the internal chamber.

The exit port can be resealable to prevent loss of fluid and/or analyte. Otherwise, a static head or capillary action can prevents fluid loss through the exit port. In another embodiment, a time period, which can be empirically determined by one of skill in the art, passes to allow analytes, such as cells, to settle by gravity to the bottom and/or to adhere to the chamber. Once the chamber is filled, preparative fluids, such as, for example, additional culture medium, can be introduced into the chamber. One of skill in the art can empirically determine the rate of fluid flow into the inlet port that is appropriate for a given analyte. The flow of the additional fluid that is added to the chamber is substantially laminar and provides for complete fluid replacement without removal of the analyte, such as cells, from the chamber. In one embodiment, the flow rate is as fast as possible without producing a shear force that disrupts and/or removes the analyte, such as cells, from the chamber. One of skill in the art can assess whether the flow rate achieves such standards by examining the output for the presence of the analyte, such as cells.

One of skill in the art will appreciate that flow rates can vary based on the material that is used to make the chamber, on the nature of the analyte, and on the type, density, viscosity, and diffusion coefficient of the fluid. In one embodiment, analytes, such as cells, can be imaged continuously or incrementally, such as before, during, or after the addition of fluids to detect cell growth, differentiation, proliferation, size, color, and/or morphology changes. Analytes, such as cells, can be stained for analysis by fluorescence microscopy, phase contrast microscopy, Nomarsky contrast microscopy, scanning electron microscopy, photography, digital imaging, and/or laser scanning cytometry.

The by-products of analytes, such as cells, may be collected, such as in the waste collection site 1110 in FIG. 11, for further analysis. Cellular by-products include, but are not limited to, antibodies, cytokines, and recombinant proteins from transfected cells. Analytes, such as magnetic particles, can be separated by the application of magnetic field.

The single flow chambers 115, provided herein, can be used in a variety of assays, including, but not limited to, biological, chemical, biochemical, and biomedical assays. Substances that can be assayed by the flow chambers provided herein include, but are not limited to, cells, lipids, crystals, magnetic beads, magnetic particles, proteins, carbohydrates, polymers, nucleic acids, and polysaccharides. In cell-based assays, the flow chambers 115 described herein can be used to assess and identify cellular response and biological activity mechanisms. The flow chambers 115 provided herein can also be used to wash efficiently adherent, partially adherent, and/or non-adherent substances with minimum use of wash fluid.

The flow chambers provided herein can be used to separate free from bound materials. The flow chambers provided herein can be used for cellular assays in which chemical and/or biochemical substances are screened for activity against cells. The chemical and/or biochemical substances include, but are not limited to, drugs, enzymes, and antibodies. The flow chambers provided herein can be used for ELISA assays. The flow chambers provided herein can be used as a cell culture chamber with a sterile environment and semi-permeable membrane for exchange of gases such as carbon dioxide and oxygen. Carbon dioxide can be used to interact with the buffering system to maintain physiological pH in cell cultures. Oxygen can be used for cellular respiration and metabolism. Living cells contained in the flow chambers provided herein can be tested or assayed for physical, chemical, and biological properties during or after cell growth using chemical, biochemical and/or immunochemical techniques. Cells that can be analyzed, screened, grown, cultured, differentiated, and/or proliferated in the flow chambers provided herein include, but are not limited to, mammalian cells, microbial cells, plant cells, insect cells, animal cells, artificial cells, recombinant cells, blood cells, human cells, transgenic cells, genetically engineered cells, transformed cells, fibroblastic cells, epithelial cells, melanoma cells, stem cells, anchorage-dependent cells, anchorage-independent cells, and tissues.

The flow chambers provided herein can be used for cell imaging in which cells that are grown in the chambers are observed with microscopy techniques and monitored for changes in response to assays or monitored for changes in comparison to untreated cells. Cells can be observed by fluoremeters, spectrophotometers, light detectors, radioactive counters, magnetometers, reflectometers, ultrasonic detectors, or by any method well known in the art. The flow chambers provided herein can be used to screen test compounds for their effect on cellular response or for their ability to stimulate, enhance, inhibit, or induce biochemical responses (e.g. biochemical responses that relate to diseases, disorders, or for symptoms of diseases and/or disorders). The flow chambers provided herein can be used to separate magnetic particles from non-magnetically coupled reagents (thus unbound to the surface of the chamber). The flow chambers provided herein can be used to assay cellular products and by-products. The flow chambers provided herein can be used for nucleic acid assays and transfection assays.

C. Device Containing a Plurality of Chambers

Provided herein are devices that include an assembly of a plurality of flow chambers 115. In an exemplary embodiment, the chambers are disposed within a support, such as a plate. For example, in one embodiment, the plate can be a microtiter plate. The support can be made from a variety of materials. Materials that can be used to make supports include, but are not limited to, plastic, thermoplastic, quartz, glass, and any other material well known in the art. The plurality of chambers 115 provided herein can have a common sealing film or a separate sealing film per chamber 115 in the assembly. The plurality of chambers 115 provided herein may be separate and distinct within the assembly, may be in fluid communication with each other, or may be connected to a general source such as, but not limited to, a manifold. The chambers 115 that are in fluid communication with each other may be connected by sterile tubing or by any apparatus known in the art to connect e.g. the outlet port of one flow chamber 115 with the inlet port 125 of another flow chamber 115.

In one embodiment, the configuration of the assembly is such that the plurality of chambers 115 is contained within a support that is the size of a standard microtiter plate. In another embodiment, the configuration of the chambers 115 within the assembly is such that it can approximate the array of wells in standard microtiter plates. In another embodiment, the configuration is such that there are at least about 24 chambers 115 in an array, at least about 96 chambers 115 in an array, at least about 384 chambers 115 in an array, at least about 864 chambers 115 in an array, at least about 1536 chambers 115 in an array, or at least about 3456 chambers 115 in an array. Provided herein are arrays of flow chambers 115 within a plate with each flow chamber 115 having at least two ports. Each flow chamber in the assembly can have at least one access port and at least one exit port in fluid communication therewith. A plurality of the flow chambers 115 can be arranged in an array disposed within a plate such as a microtiter plate, or in some embodiments, the flow chambers may be arranged in an alternative configuration. An array of chambers can be manufactured to comply with the Society for Biomolecular Screening Microplate Standards for microtiter plate geometries.

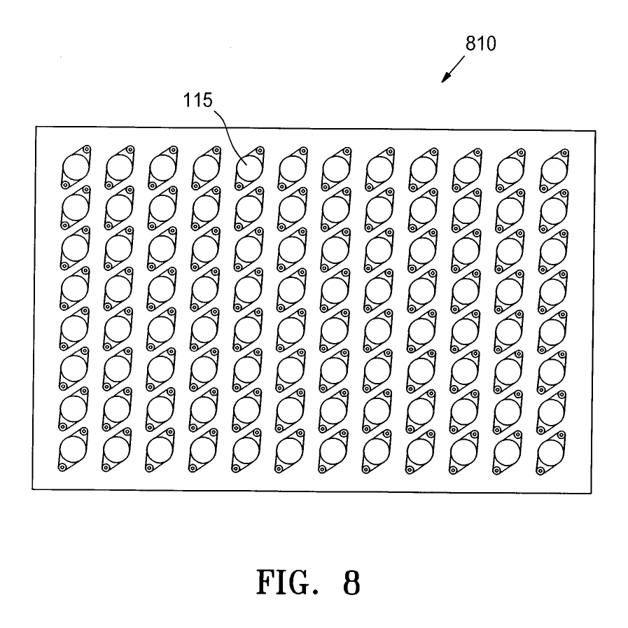

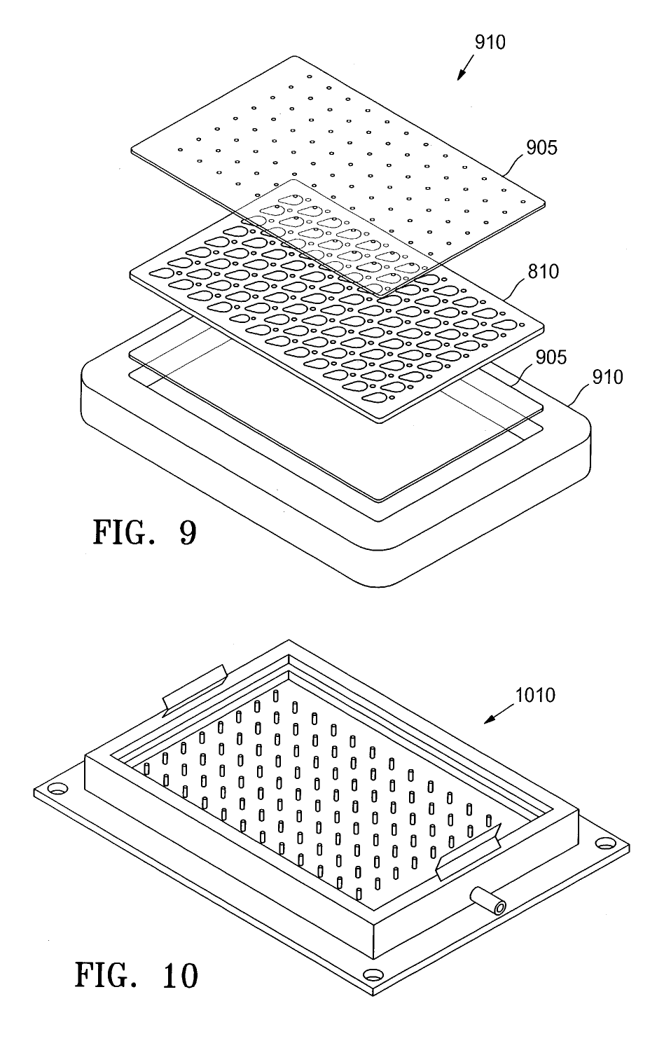

FIG. 8 shows a top view of a plate 810 containing an array of chambers 115. For ease of illustration in FIG. 8, only a single flow chamber 115 is labeled with the reference numeral 115, although the plate 810 includes an array of chambers 115 arranged in a 12×8 grid. In an embodiment as shown in the exploded view of an assay device 910 in FIG. 9, the chamber plate 810 has an array of chambers 115 which are sealed on upper and lower sides with a sealing film 905 made of, for example, a transparent membrane such as polyolefin. Other materials well known in the art can be used as a sealing film. Non-transparent material can be used to make the sealing film, especially when interrogation of the titrant or cellular by-product is the objective of the assay and/or experiment. For embodiments of an assay device with the exit port in an open configuration (i.e., uncovered by a sealing film), surface tension of fluid within the exit port can prevent escape of flow chamber fluid in the absence of injection pressure into the access port.

FIG. 9 illustrates a rectangular array of flow chambers which are disposed within a frame 910. The frame can be made from polystyrene, cycloolefin, or any other suitable medical grade polymer. The sealing film 905 can be a gas permeable transparent sheet of material or can be made of alternate suitable materials which may or may not be gas permeable. The array can have a footprint of an industry standard microtiter plate with the chambers 115 and access ports disposed within the array to provide a standardized spacing between chambers that can allow for use with existing commercial automation equipment. Arrays of these chambers, such as an 8×12 array, can be used with industry-standard liquid handling manifolds for processing of the contents within the chamber simultaneously. Commercial pipetting manifolds use polymer nozzles or tips that seal with pressure into the taper of the access port. In an embodiment of the device with a flow-through chamber design, the pipettor can act as both a dispenser and washer in that an injection of a fluid of appropriate volume into the access port can displace the existing volume of the contents of the chamber. FIG. 10 shows a waste adapter 1010 which is in fluid communication with the exit port of one or more flow chambers which may be in an array contained on a plate as described above.

Embodiments of an assay device can include an array of flow chambers 115 disposed in a rectangular plate. The chambers can be arranged in a 4×6 array, 8×12 array, 16×24 array, or any other suitable configuration or multiple thereof. The flow chambers 115 of the array can have two ports each, an inlet port or an access port for introduction of fluids or other materials into the chamber which can be pipette tip compatible and an outlet port or drain port for exit of fluids or other materials as described above with regard to FIGS. 1-7, 11, and 12. The dimensions of some embodiments of the assay device can be configured to comply with the Society for Biomolecular Screening Microplate Standards.

1. Preparation of Devices Containing a Plurality of Chambers

The devices containing a plurality of chambers can be made by injection molding using methods well known in the art. A mold of an array of chambers can be made from plastic or from several pieces of plastic that are joined together. In particular, one of skill in the art can make clam shells via plastic injection molding and laminate clear plastic sealing film on one or both sides via solvent welding, sonic welding, heat, or glue. A skirt can be added to give the chambers the general appearance of a microtiter plate, and sealing film placed individually on each chambers or one film placed commonly on the chambers. The dimensions of the array of chambers can be configured to comply with the Society for Biomolecular Screening Microplate Standards. The array of chambers in the Microplate-standard geometry can be in the format of 96 chambers in an 8×12 grid or 384 chambers in 16×24 grid or any other size, spacing, and layout.

2. Use of Devices Containing a Plurality of Chambers

Arrays of chambers can be used in the same manner as described herein for single chambers. Arrays of chambers provide for high throughput screening and assays and for parallel handling of assays and can be adapted to automated processes using methods well known in the art.

D. Methods

Provided herein are methods for observing, monitoring and/or analyzing a plurality of cells, e.g., a culture or colony of cells, and/or processes involving a plurality of cells within a flowing fluid environment wherein the fluid flow is substantially laminar, such as using a flow cell 110 or an array of flow cells 110. Also provided are methods of using an apparatus containing one or more flow chambers 115 as described herein. The flow chambers 115 provided herein can be used, for example, to conduct cellular assays or to study biological and non-biological mechanisms. Cellular assays include, but are not limited to, stabilizing cells, treating cells with chemical and/or physical challenges, waiting for the cells to respond to the chemical and/or physical challenges, and measuring and/or observing the reaction of e.g. cells to the chemical and/or physical challenges. Cells that can be assayed in the flow chambers include, but are not limited to, mammalian cells, microbial cells, plant cells, insect cells, animal cells, yeast cells, CHO cells, hepatocytes, stem cells, neurons, artificial cells, recombinant cells, blood cells, human cells, transgenic cells, genetically engineered cells, transformed cells, fibroblastic cells, epithelial cells, melanoma cells, anchorage-dependent cells, anchorage-independent cells, tissues, and multi-cellular organisms. Cellular response to chemical and/or physical challenges can be observed and/or measured by methods such as, but not limited to, adding reagents (e.g. titrants), visual observation, microscopy, and treatment with electromagnetic waves. Biological mechanism that can be studied include, but are not limited to, ligand-receptor recognition, nucleic acid hybridization, transfection, and enzymatic catalysis. Non-biological mechanisms include, but are not limited to, magnetic particle separation of selected analytes from samples. The cell-based assays, biological and non-biological mechanisms can be conducted in a single flow chamber or on an array of flow chambers, such as, but not limited to, an array of flow chambers on standard microtiter plates with 96, 384, 864, 1536, or 3456 wells. The cell-based assays, biological and non-biological mechanisms conducted in single flow chambers or on arrays of flow chambers can be automated using methods well known in the art. Provided herein are methods of using the flow chambers provided herein for cell imaging, magnetic particle separation, ELISA, nucleic acid assays, cell culturing, transfection assays, and cell-based assays.

1. Cell Imaging Assay

Embodiments of the assay device provided herein provide for a cellular imaging method which can involve, “Preparation” and “Processing” followed by “Imaging”, as outlined below. The dispensing of fluid through the chambers as described in the present embodiment can avoid the need for a plate washer and can allow for the retention of barely adherent cells during dispensing and washing. The cell-based imaging assay can be conducted in a single chamber or on an array of chambers and can be adapted for automated processes.

a. Preparation

In one embodiment, a suspension of cells is dispensed into an empty flow chamber 115. The cells are incubated in the flow chamber for a required period of time as determined by one of skill in the art. A compound of interest that has been diluted in an appropriate media is dispensed into the flow chamber, displacing the supernatant. A compound of interest can include, but is not limited to, an antigen, an antibody, a ligand, a drug, a protein, an enzyme, a nucleic acid, a carbohydrate, a lipid, a reagent, a titrant, a pH indicator, an inorganic compound, and an organic compound. The contents of the flow chamber are incubated for an appropriate time period as determined by one of skill in the art. A fixative is dispensed into the flow chamber, displacing the supernatant. A fixative can make cells permeable to staining reagents and can crosslink the macromolecules of cells so that they are stabilized and locked into position.

b. Processing

A blocking agent is dispensed into the flow chamber 115, displacing the supernatant. A first stain is dispensed into the flow chamber, displacing the supernatant. A second stain is dispensed into the flow chamber, displacing the supernatant. A third stain is dispensed into the flow chamber, displacing the supernatant. A buffer is dispensed into the flow chamber, displacing the supernatant.

C. Imaging

The flow chamber 115 is loaded onto an automated imaging workstation, and an image of at least some of the contents of the flow chamber is obtained. The contents of the flow chamber can be imaged using methods such as, but not limited to, microscopy, in which cells are greater than 50 μm², greater than 100 μm² to about 1000 μm² image within the microscope field of view. In another embodiment, the cell-imaging assay can be carried out on an array of flow chambers disposed on a plate and can be adapted for automated processes.

2. Magnetic Particle Separation Assay

Provided herein is a method of using the flow chambers provided herein for magnetic particle separation. The magnetic particle assay can be conducted in a single chamber or on an array of chambers and can be adapted for automation processes.

In one embodiment, a fluid containing a suspension of mixed population of cells including the target cells to be isolated from the mixture is introduced into the inlet port of the chamber via a pipette tip. A solution and/or suspension of magnetic separation reagent coated with a ligand that has a binding specificity and affinity for the target cells is added to the flow chamber via pipette. Magnetic separation reagents include, but are not limited to, commercially available magnetic beads (e.g. Dynal beads) coated with antibodies. After a sufficient time to allow for the magnetic separation reagent and target cells to bind, a magnetic field is applied to separate and/or pull away the target cells bound by the magnetic reagent from the mixture of cells. The magnetic field can be supplied by a magnet, a ferromagnetic material, magnetic powder (e.g. barium ferrite), or any magnetic material known to those of skill in the art. A wash is used to completely displace non-tagged cells from the chamber while the magnetically bound target cell-magnetic separation reagent remains in the chamber.

The target cells can be further visualized by methods such as, but not limited to microscopy, after the separation step. In an alternative embodiment, a mixture of cells is pre-treated with the magnetic separation reagent before introduction into the flow chamber. In another embodiment, the magnetic separation reagent has a fluorescent label for visualization of the magnetic particle-tagged cells after separation. In another embodiment, the magnetic separation reagent is removed after the separation of the cells. In another embodiment, a trypsin detaching reagent is used to resuspend cells in the medium prior to magnetic separation.

Another embodiment of an assay device can provide for use of the device in magnetic particle based assay methods which can involve the procedures of “Preparation” and “Reading”, as outlined below.

a. Preparation

A magnetic particle suspension is dispensed into an array of empty flow chambers 115 on a plate 810. A magnetic field is applied to capture particles in the flow chambers of the plate. A sample is dispensed into the flow chambers, displacing the supernatant. The magnetic field is removed, and the plate is externally agitated. The contents of the flow chambers within the plate are incubated for an appropriate period of time as determined by one of skill in the art. A magnetic field is applied to the plate. A buffer is dispensed into the flow chambers, collecting the supernatant into a stacked empty plate, or, alternatively, a buffer is dispensed into the flow chambers, displacing the supernatant.

b. Reading

The plate 810 containing the array of flow chambers 115 is loaded onto a plate reader, and the read is initiated. A cleaving reagent is dispensed into the flow chambers, displacing the supernatant. The contents of the flow chambers are incubate for a period of time as determined by one of skill in the art. A buffer is dispensed into the flow chambers, and the supernatant of the flow chambers is collected into a stacked empty plate. In an alternative embodiment, the magnetic particle separation assay is conducted on a single flow chamber and is adapted for automated processes.

3. ELISA-based Assay

ELISA (Enzyme-linked immunosorbent assay) is an assay that can be used to quantitate the amount of antigen, proteins, or other molecules of interest in a sample. In particular, ELISA can be carried out by attaching on a solid support (e.g. polyvinylchloride) an antibody specific for an antigen or protein of interest. Cell extract or other sample of interest can be added for formation of an antibody-antigen complex, and the extra, unbound sample is washed away. An enzyme-linked antibody, specific for a different site on the antigen is added. The support is washed to remove the unbound enzyme-linked second antibody. The enzyme-linked antibody can include, but is not limited to, alkaline phosphatase. The enzyme on the second antibody can convert an added colorless substrate into a colored product or can convert a non-fluorescent substrate into a fluorescent product. The ELISA-based assay method provided herein can be conducted in a single chamber or on an array of chambers and can be adapted for automation processes.

Embodiments of an assay device having some or all of the features discussed above can be used in an ELISA based assay which involves the following procedures of “Preparation” followed by “Reading”.

a. Preparation

A sample-labeled analyte complex is introduced into a flow chamber 115 by pipette or other means well known in the art. The sample can include, but is not limited to, a cell and/or cell extract, and the labeled analyte can include, but is not limited to, an antibody. The contents of the flow chamber are incubated for a period of time that can be determined by one of skill in the art. A preparative fluid, such as a buffer, is dispensed into the flow chamber, and the supernatant is displaced. An enzyme substrate reagent is added to the flow chamber, and the supernatant is displaced. An enzyme substrate reagent can include, but is not limited to, an enzyme-linked second antibody. The contents of the flow chamber are incubated for a period of time that can be determined by one of skill in the art, and a preparative fluid, such as stop solution, is added to the flow chamber followed by collection of the supernatant onto a stacked empty plate. In an alternative embodiment, the labeled analyte, such as an antibody, is introduced into the flow chamber first followed by introduction of the sample and an incubation period.

b. Reading

The flow chamber 115 is loaded onto a plate reader for initiation of the reading and observation of results. Observation of results can be carried out using microscopy, fluorescence microscopy, or by any method well known in the art.

4. Nucleic Acid Assay

Assay devices having some or all of the features discussed above can be used in a nucleic acid assay such as the Molecular Beacon Assay by Promega which can involve the following procedures of “Preparation” followed by “Reading”. Any binding detection assay using hybridization of nucleic acid sequences with fluorescent detection through quenching or double strand nucleic acid antibodies can be used.

a. Preparation

A sample/labeled analyte is dispensed into empty flow chambers of a plate. The contents of the flow chamber or the plate containing the array of flow chambers are incubated for a period of time, as determined by one of skill in the art, for hybridization to occur. A buffer is dispensed into the flow chambers, displacing the supernatant. A substrate reagent is dispensed into the flow chambers, displacing the supernatant.

b. Reading

The plate containing the array of flow chambers is loaded onto a plate fluorescence reader, and the read is initiated. In an alternative embodiment, the nucleic acid assay is conducted on a single flow chamber and is adapted for automated processes.

5. Cell Culture Assay

A method of culturing cells in the flow chambers provided herein is provided. The cell culturing assay can be conducted in a single chamber or an array of chambers and can be adapted for automation processes.

Cells to be cultured are suspended in a sufficient amount of culture medium to support cell growth. Thus suspension containing the cells of interest are introduced into the flow chamber via a pipette tip that is inserted into the inlet port. The cells are allowed to settle by gravity into the chamber and are allowed to attach to the chamber. Fresh culture medium is introduced via pipette tip into the inlet port to completely displace the supernatant. The cells are allowed to incubate using methods well known in the art. The growth of cells are observed via the imaging site using methods, such as, but not limited to, microscopy and fluorescence microscopy. The cell culturing may occur with a semi-permeable transparent membrane for proper exchange of gases, or the culturing may include a venting step. In the venting step, air or gas is displaced from the flow chamber when cells or the culture medium is introduced into the flow chamber. In one embodiment, the same inlet port is used for the introduction of cells and/or culture medium and for allowing air or gas (e.g. bubbles) to be withdrawn from the flow chamber. In another embodiment, another port is used for venting. In an alternative embodiment, more than one inlet port is used for introducing cells and/or culture medium into the chamber and for allowing gas, air, or a solution to be displaced from the flow chamber.

6. Transfection Assay

Provided herein is a method of using the flow chambers provided herein for transfection assays. The transfection assay can be conducted in a single chamber or on an array of chambers and can be adapted for automated processes. A suspension of cells is added to the flow chamber via a pipette tip. The cells are allowed to settle by gravity. A solution of fresh culture medium is added to the flow chamber. In a tube, a solution of a plasmid that encodes a green fluorescent protein, a lipid-containing transfection reagent, and culture medium is allowed to incubate. The mixture of transfection reagent, medium, and plasmid is introduced into the flow chamber to react with adherent or partially adherent cells. The flow chamber is then incubated using methods well known in the art. A solution of fresh culture medium is added to the flow chamber to remove the transfection reagent mixture. The transfected cells are then analyzed and quantitated for fluorescence produced by the green fluorescent protein.

7. Cell-based Assay

Provided herein is a method of using the flow chambers provided herein for cell-based assays. The cell-based assays can be used for purposes, such as, but not limited to, screening for activity against cells, screening test compounds, for studying enzymatic catalysis, for studying ligand-receptor recognition, and for assaying and/or harvesting cell products and by-products. The cell-based assay can be conducted in a single chamber or on an array of chambers and can be adapted for automated processes.

A suspension of cells is dispensed into a flow chamber via pipette tip. The cells are allowed to settle by gravity into the chamber and are allowed to attach to the chamber. Fresh medium is dispensed into the flow chamber, displacing the supernatant. The cells are allowed to incubate for a time period as determined by one of skill in the art. A solution of a preparative fluid, such as test compound(s), is dispensed into the flow chamber, displacing the supernatant. The fluid flows laminarly through the flow chamber. In an alternative embodiment, a solution of a ligand(s) is dispensed into the flow chamber, displacing the supernatant. In another embodiment, solution of an enzyme(s) is dispensed into the flow chamber, displacing the supernatant. The contents of the flow chamber are allowed to incubate for a time period as determined by one of skill in the art. In one embodiment, a buffer is dispensed into the flow chamber, displacing the supernatant, and the supernatant is assay for cell products and by-products. In another embodiment, cellular response to the test compound, ligand, and/or enzyme is observed for size, differentiation, proliferation, and/or morphological changes in response to the test compound, ligand, and/or enzyme via such methods as, but not limited to, microscopy and fluorescence microscopy.

Although embodiments of the present device and methods of use thereof are described in detail with reference to certain versions, other versions, embodiments, methods of use, and combinations thereof are also possible. Therefore the spirit and scope of the appended claims should not be limited to the description of the embodiments contained herein.

What is claimed is:

1. A flow chamber for conducting a biological process, comprising;
a fluid enclosure including one or more walls that are disposed about and substantially enclose a chamber portion through which fluid can flow along a flow path;
an inlet port in fluid communication with the chamber portion;
an outlet port in fluid communication with the chamber portion;
wherein the chamber portion has a cross-sectional area along a plane that is orthogonal to the fluid flow path, and wherein the cross-sectional area of the chamber portion is constant or changes smoothly and continuously moving along the flow path, and wherein at least dimension of the cross-sectional area is greater than about 500 μm.

2. The flow chamber of claim 1, wherein the chamber portion has a substantially constant cross sectional area along the flow path.

3. The flow chamber of claim 1, wherein the chamber portion has one or more changes in cross sectional area along the flow path.

4. The flow chamber of claim 1, further comprising an inlet conduit disposed between and in fluid communication with the inlet port and the chamber portion.

5. The flow chamber of claim 1, further comprising an outlet conduit disposed between the chamber section and the outlet port and in fluid communication with the outlet port and the chamber section.

6. The flow chamber of claim 1, further comprising a waste reservoir that collects fluid that flows out of the outlet port.

7. The flow chamber of claim 1, wherein an interrogation surface is located on one of the walls inside the chamber portion.

8. The flow chamber of claim 7, wherein at least a portion of the interrogation surface is at least partially transparent.

9. The flow chamber of claim 8, wherein the interrogation surface is an imaging site viewable through the transparent portion of the interrogation surface.

10. The flow chamber of claim 7, wherein the interrogation surface is non-transparent.

11. The flow chamber of claim 1, wherein at least one of the walls comprises a membrane that seals the fluid enclosure.

12. The flow chamber of claim 11, wherein the membrane is transparent.

13. The flow chamber of claim 11, wherein the membrane is gas permeable.

14. The flow chamber of claim 1, wherein the inlet and outlet ports are on opposite sides of the flow chamber.

15. The flow chamber of claim 1, wherein the inlet and outlet ports are on the same side of the flow chamber.

16. The flow chamber of claim 1, wherein the chamber portion is configured to facilitate a flow of fluid that is substantially laminar.

17. The flow chamber of claim 7, wherein the chamber portion is configured to facilitate a substantially laminar flow of fluid across the interrogation surface.

18. The flow chamber of claim 1, wherein the chamber portion has an internal volume capacity of about 1 to about 1000 μL.

19. The flow chamber of claim 18, wherein the chamber portion has an internal volume capacity of about 70 to about 100 μL.

20. The flow chamber of claim 19, wherein the chamber portion has an internal volume capacity of about 35 to about 45 μL.

21. The flow chamber of claim 1, wherein the chamber portion has an internal volume capacity of about 15 to about 25 μL.

22. A plurality of flow chambers disposed in an ordered array with each flow chamber comprising:
a fluid enclosure including one or more walls that are disposed about and substantially enclose a chamber portion through which fluid can flow along a flow path;
an inlet port in fluid communication with the chamber portion;
an outlet port in fluid communication with the chamber portion;
wherein the chamber portion has a cross-sectional area along a plane that is orthogonal to the fluid flow path, and wherein the cross-sectional area of the chamber portion is constant or changes smoothly and continuously moving along the flow path, and wherein at least dimension of the cross-sectional area is greater than about 500 μm.

23. The plurality of flow chambers of claim 22, wherein the chamber portion of each flow chamber has an internal volume capacity of about 1 to about 1000 μL.

24. The plurality of flow chambers of claim 22, wherein the chamber portion of each flow chamber has an internal volume capacity of about 70 to about 100 μL.

25. The plurality of flow chambers of claim 22, wherein the chamber portion of each flow chamber has an internal volume capacity of about 35 to about 45 μL.

26. The plurality of flow chambers of claim 22, wherein the chamber portion of each flow chamber has an internal volume capacity of about 1 to about 10 μL.

27. The plurality of flow chambers of claim 22, wherein the array is comprised of one or more flow chambers disposed within a microtiter plate.

28. The plurality of flow chambers of claim 22, comprised of 96 chambers in an 8 by 12 array disposed within a microtiter plate.

29. A method for performing a cell imaging assay comprising:
a) providing a flow chamber comprising:
a fluid enclosure including one or more walls that are disposed about and substantially enclose a chamber portion through which fluid can flow along a flow path;
an inlet port in fluid communication with the chamber portion;
an outlet port in fluid communication with the chamber portion;
wherein the chamber portion has a cross-sectional area along a plane that is orthogonal to the fluid flow path, and wherein the cross-sectional area of the chamber portion is constant or changes smoothly and continuously moving along the flow path, and wherein at least dimension of the cross-sectional area is greater than about 500 μm;
b) dispensing a cell suspension into the flow chamber;
c) incubating contents of the flow chamber;
d) dispensing preparative fluids into the flow chamber such that the flow of the fluid is substantially laminar within the flow chamber;
e) imaging and observing the cells on the interrogation surface of the flow chamber.

30. The method of claim 29, further comprising incubating the contents of the flow chamber following dispensing the preparative fluids into the flow chamber.

31. The method of claim 29, wherein the preparative fluids include a fixative solution, a blocking agent, a first stain, a second stain, a third stain, and a buffer.

32. The method of claim 29, wherein in step d), the preparative fluid displaces another preparative fluid.

33. The method of claim 29, wherein the preparative fluid flow through the chamber at a rate of about 8 μl/s to about 43 μl/s.

34. A method for performing an ELISA-based assay comprising:
a) providing a flow chamber comprising a fluid enclosure which is disposed about and substantially encloses a chamber portion;
a fluid enclosure including one or more walls that are disposed about and substantially enclose a chamber portion through which fluid can flow along a flow path;
an inlet port in fluid communication with the chamber portion;
an outlet port in fluid communication with the chamber portion;
wherein the chamber portion has a cross-sectional area along a plane that is orthogonal to the fluid flow path, and wherein the cross-sectional area of the chamber portion is constant or changes smoothly and continuously moving along the flow path, and wherein at least dimension of the cross-sectional area is greater than about 500 μm;
b) dispensing a sample into the flow chamber;
c) incubating the contents of the flow chamber;
d) dispensing a first preparative fluid into the flow chamber such that the flow of fluid is substantially laminar;
e) dispensing an enzyme substrate reagent into the flow chamber such that the flow of fluid is substantially laminar;
f) incubating the contents of the chamber;
g) dispensing a second preparative fluid into the flow chamber such that the flow of fluid is substantially laminar, such that the supernatant is displaced, and such that the sample is retained within the flow chamber;
h) analyzing the sample or supernatant.

35. The method of claim 34, wherein the preparative fluids include a buffer and a stop solution.

36. The method of claim 34, wherein in step d), the first preparative fluid can displace another preparative fluid.

37. The method of claim 34, wherein the preparative fluid flows through the chamber at a rate of about 8 μl/s to about 43 μl/s.

38. The method of claim 34, wherein the flow chambers are arranged in an array.

39. A method for performing a cell culture assay comprising:
a) providing a flow chamber comprising:
a fluid enclosure including one or more walls that are disposed about and substantially enclose a chamber portion through which fluid can flow along a flow path;
an inlet port in fluid communication with the chamber portion;
an outlet port in fluid communication with the chamber portion;
wherein the chamber portion has a cross-sectional area along a plane that is orthogonal to the fluid flow path, and wherein the cross-sectional area of the chamber portion is constant or changes smoothly and continuously moving along the flow path, and wherein at least dimension of the cross-sectional area is greater than about 500 μm;
b) dispensing into the flow chamber a suspension of cells to be cultured;
c) incubating contents of the flow chamber;
d) dispensing into the flow chamber preparative fluids such that the flow of the preparative fluid is substantially laminar;
e) incubating the contents of the flow chamber to effect growth of the cells contained therein; and
f) observing growth, differentiation, proliferation, and/or morphology of the cells.

40. The method of claim 39, wherein the preparative fluids include a buffer, fluorescent dye, and a culture medium.

41. The method of claim 39, wherein in step d), the preparative fluid can displace another preparative fluid.

42. The method of claim 39, wherein the preparative fluid flows through the chamber at a rate of about 8 μl/s to about 43 μl/s.

43. A method for performing a transfection assay comprising:
a) providing a flow chamber comprising:
a fluid enclosure including one or more walls that are disposed about and substantially enclose a chamber portion through which fluid can flow along a flow path;
an inlet port in fluid communication with the chamber portion;
an outlet port in fluid communication with the chamber portion;
wherein the chamber portion has a cross-sectional area along a plane that is orthogonal to the fluid flow path, and wherein the cross-sectional area of the chamber portion is constant or changes smoothly and continuously moving along the flow path, and wherein at least dimension of the cross-sectional area is greater than about 500 μm;
b) dispensing a suspension of cells into the flow chamber;
c) incubating the contents of the flow chamber;
d) dispensing into the flow chamber preparative fluids such that the flow of the preparative fluid is substantially laminar; and
e) analyzing the contents of the flow chamber.

44. The method of claim 43, wherein the preparative fluids include a buffer, fluorescent dye, a plasmid solution, nucleic acid solution, a transfection reagent, a wash solution, and a culture medium.

45. The method of claim 43, wherein in step d), the preparative fluid can displace another preparative fluid.

46. The method of claim 43, wherein the preparative fluid flows through the chamber at a rate of about 8 μl/s to about 43 μl/s.
